(19) United States
(12) Patent Application Publication
(10) Pub. No.: US 2014/0242726 A1
(43) Pub. Date: Aug. 28, 2014

(54) LUNG CANCER MARKER COMPLEMENT C3dg MOLECULE, AND METHOD FOR ANALYZING LUNG CANCER MARKER

(71) Applicants: Atsuhiko Toyama, Toshima-ku (JP); Taka-Aki Sato, Shinagawa-ku (JP); Koji Ueda, Setagaya-ku (JP)

(72) Inventors: Atsuhiko Toyama, Toshima-ku (JP); Taka-Aki Sato, Shinagawa-ku (JP); Koji Ueda, Setagaya-ku (JP)

(21) Appl. No.: 14/343,895

(22) PCT Filed: Mar. 19, 2012
(86) PCT No.: PCT/JP2012/057050

§ 371 Date: Mar. 25, 2014

(30) Foreign Application Priority Data
Sep. 15, 2011 (JP) 2011-201380

(51) Int. Cl. G01N 33/574

(52) U.S. Cl. CPC G01N 33/57423
USPC 436/501; 530/395

(57) ABSTRACT

[Problem]

To provide a method for detecting early-stage lung cancer, which reduce a burden on a subject and are excellent in specificity and sensitivity. [Solution]

A lung cancer marker comprising a complement C3dg molecule. A method for analyzing a lung cancer marker comprising the steps of: measuring a level of a complement C3dg molecule as a lung cancer marker in a biological sample to acquire a measured level of the complement C3dg molecule; and making an evaluation as to whether the measured level is high or low based on a reference level of the complement C3dg molecule. Preferably, the measured level of the complement C3dg molecule is a value relative to a measured level of a complement C3 full-length molecule in the biological sample, and herein the reference level of the complement C3dg molecule is a value relative to a reference level of the complement C3 full-length molecule.

### TECHNICAL FIELD

The present invention relates to screening for lung cancer, and particularly to a lung cancer marker usable for early diagnosis.

### BACKGROUND ART

Lung cancer is the leading cause of death from cancer in Japan, Europe, and the United States, and is refractory. Lung cancer is divided into small-cell lung cancer and non-small cell lung cancer, and small-cell lung cancer accounts for 15% of the total number of lung cancer patients and non-small cell lung cancer accounts for the remaining 85%. Further, non-small cell lung cancer can be histopathologically divided into three types: adenocarcinoma, squamous cell carcinoma, and large-cell cancer.

Smoking remains the main risk factor of cancer. However, lung cancer (mainly adenocarcinoma) in non-smokers has recently increased. The five-year survival rate for lung cancer patients is only about 15%. This largely results from the fact that there is no method capable of detecting lung cancer at an early stage. Cases to be diagnosed with early-stage disease of the lung cancer patients are only 16%. Chest radiography, sputum cytology, helical CT and the like have been used as screening tools for lung cancer detection, but have had little effect on improving the mortality rate for lung cancer.

On the other hand, serum biomarkers for lung cancer have been developed in order to achieve early detection of disease and improve clinical management (Non-Patent Document 1). However, their clinical usefulness is limited (Non-Patent Document 2) . The amounts of CEA (carcinoembryonic antigen) and CYFRA (cytokeratin 19 fragment) present in serum increase in some of small-cell lung cancer patients. Therefore, they are clinically effective in monitoring disease status or evaluating response to therapy (Non-Patent Document 3).

In recent years, it has become possible to find a novel biomarker having potential to be used for cancer diagnosis by monitoring protein expression patterns in clinical specimens using proteomics technologies. In order to deepen the understanding of the molecular basis of cancer pathogenesis and the characteristics of cancer-related proteins, various proteomics tools such as 2D-DIGE (2 Dimensional Fluorescence Difference Gel Electrophoresis), SELDI-TOF MS (surface-enhanced laser desorption/ionization time-of-flight mass spectrometry), protein array, ICAT (Isotope-coded affinity tags), iTRAQ (Isobaric tags for relative and absolute quantification), and MudPIT (Multidimensional Protein Identification Technology) have been used for differential analysis of various biological samples (Non-Patent Document 4). The biological samples include cell lysates, serum, plasma and the like. Similarly, fragments generated by protein processing or degradation occurring characteristic of a disease are also promising candidates for biomarkers (Non-Patent Document 5).

On the other hand, complement component 3 (C3) is known as a complement component. Human complement C3 is a glycoprotein having a molecular weight of about 180 kDa and a structure in which an α chain and a β chain are cross-linked by a S—S bond, and the amount of human complement C3 contained in blood is the largest among complement components. Human complement C3 is concerned in the activation of the complement system. It is known that, in some types of cancer, the amount of human complement C3 present in blood increases with advance of cancer. For example, JP-A-2007-51880 (Patent Document 1) discloses a complement C3 precursor as a candidate for a pancreas cancer biomarker.

### PRIOR ART DOCUMENTS

### Non-Patent Documents

Non-Patent Document 1: Journal of the National Cancer Institute, 1988, vol. 80, pp. 97-101

Non-Patent Document 2: Chest, 2002, vol. 122, pp. 1037-1057

Non-Patent Document 3: BMB Reports, 2008, vol. 41, pp. 615-625

Non-Patent Document 4: Anticancer Research, 2007, vol. 27, pp. 1247-1255

Non-Patent Document 5: PLos One, vol. 4, e7731, [online], November 5, 2009, [searched on September 15, 2011], Internet<URL:http://www.plosone.org/article/fetchArticle.action?articleURI=info:doi/10.1371/journal.pone.0007731>

### Patent Document

Patent Document 1: JP-A-2007-51880

### SUMMARY OF THE INVENTION

### Problems to be Solved by the Invention

CEA and CYFRA are regarded as being clinically effective, but are not suitable for use in diagnosis of lung cancer. This is because it is known that CEA and CYFRA are related also to smoking, other diseases such as pneumonia, and other types of cancer, and in addition, cannot detect early-stage lung cancer.

Further, none of biomarker candidates that have been found so far using proteomics technologies are practically used as lung cancer biomarkers. The main reason for this is considered to be that these biomarker candidates are poor in specificity or sensitivity.

Further, the complement C3 precursor is known as a candidate for a pancreas cancer biomarker, but it is also known that the amount of complement C3 in blood varies due to other factors such as immunological response and hepatic dysfunction. That is, it is assumed that complement C3 does not function alone as a biomarker for determination of cancerous diseases.

Existing blood tumor markers are poor in sensitivity and specificity, and therefore cannot be expected to have the effect of detecting lung cancer with high accuracy. On the other hand, image diagnosis and various detailed examinations are all direct methods, but such methods with higher reliability are more invasive and impose a greater burden on a subject. Therefore, an object of the present invention is to provide a method for detecting early-stage lung cancer and a lung cancer diagnosis kit which reduce a burden on a subject and are excellent in specificity and sensitivity.

### Means for Solving the Problems

The present inventors have newly found that a complement C3dg fragment, which is present in a certain amount in biological samples of normal subjects, is significantly reduced in biological samples of subjects with early-stage lung cancer, which has led to the completion of the present invention.

The present invention includes the following aspects.

(1) A lung cancer marker comprising a complement C3dg molecule.
(2) A method for analyzing a lung cancer marker comprising the steps of:

measuring a level of a complement C3dg molecule as a lung cancer marker in a biological sample to acquire a measured level of the complement C3dg molecule; and

making an evaluation as to whether the measured level is high or low based on a reference level of the complement C3dg molecule.

(3) The method according to (2), wherein the measured level of the complement C3dg molecule is a value relative to a measured level of a complement C3 full-length molecule in the biological sample, and

wherein the reference level of the complement C3dg molecule is a value relative to a reference level of the complement C3 full-length molecule.

(4) The method according to (2) or (3) , further comprising the step of subjecting the biological sample to a separation step to obtain the separated complement C3dg, wherein in the step of acquiring the measured level of the complement C3dg molecule, the complement C3dg molecule is measured using an antibody whose epitope is at least all or part of a complement C3dg sequence.
(5) The method according to (3), wherein in the step of acquiring the measured level of the complement C3dg molecule, the complement C3 full-length molecule and the complement C3dg molecule are detected at the same time by mass spectrometry to determine an amount of the detected complement C3dg molecule relative to an amount of the detected complement C3 full-length molecule.
(6) The method according to (2), wherein in the step of acquiring the measured level of the complement C3dg molecule, the complement C3dg molecule is detected by mass spectrometry.

In the method according to the above (6), the detection of a complement C3 full-length molecule does not need to be performed as in the method according to the above (5).

(7) A lung cancer detection kit comprising an anti-complement C3dg antibody.

### Effects of the Invention

According to the present invention, it is possible to provide a method for detecting early-stage lung cancer and a lung cancer diagnosis kit which reduce a burden on a subject and are excellent in specificity and sensitivity.

### BRIEF DESCRIPTION OF THE DRAWINGS

FIG. 1 shows the results of C3dg molecule detection obtained by subjecting serum samples to western blotting.

FIG. 2 is a graph obtained by plotting C3dg molecule relative expression level detected in FIG. 1.

### MODES FOR CARRYING OUT THE INVENTION

### [1. Lung Cancer Marker]

The present invention provides a lung cancer marker.

A complement C3dg molecule provided as the lung cancer marker according to the present invention has been found in serum samples of both lung cancer patients and normal subjects. Specifically, the complement C3dg molecule has been found by subjecting serum samples of lung cancer patients and normal subjects to immunodepletion, deglycosylation, tryptic digestion, and LC-MALDI MS measurement, performing two-dimensional mapping of the measurement results to quantify peaks and perform statistical analysis, and then performing peptide identification by retrospective MS/MS and verification by MRM (Multiple Reaction Monitoring) and western blotting.

The complement C3dg molecule as the lung cancer marker according to the present invention is generated by stepwise degradation and fragmentation of complement component 3 (C3), which is a complement component, in a living body, and is specifically a fragment molecule having an amino acid sequence from position 955 to position 1303 of complement C3. More specifically, the complement C3dg molecule comprises an amino acid sequence represented by SEQ ID No. 1. Further, when an individual as a source of a biological sample to be subjected to detection of the complement C3dg molecule has polymorphic or allelic variation including substitution, deletion, insertion, or addition of one or two or more amino acids or a combination thereof in the amino acid sequence represented by SEQ ID No. 1, the complement C3dg molecule is the amino acid sequence represented by SEQ ID No. 1 with said polymorphic or allelic variation.

The complement C3dg molecule has a molecular weight of about 39 kDa. More specifically, the molecular weight is 38, 905 Da, which is a theoretical value determined from SEQ ID No. 1. However, the measured molecular weight may slightly vary depending on the type of method or instrument used for measurement. For example, when mass spectrometry is used, the measured molecular weight of the complement C3dg molecule may be the theoretical value ±0.5% (preferably ±0.3%, and more preferably ±0.1%).

The amount of the complement C3dg molecule contained in biological samples of lung cancer patients, especially of early-stage lung cancer patients, is significantly reduced as compared to biological samples of normal subjects.

Therefore, the complement C3dg molecule as the lung cancer marker according to the present invention can distinguish between lung cancer patients and normal subjects. Particularly, the lung cancer marker according to the present invention can distinguish between early-stage lung cancer patients and normal subjects. The lung cancer marker according to the present invention can be used for screening for lung cancer, especially for early diagnosis of lung cancer.

### [2. Method for Analyzing Lung Cancer Marker]

The present invention provides a method for analyzing a biological sample with the use of a complement C3dg molecule as a lung cancer marker. The method according to the present invention uses a complement C3dg molecule as a marker whose expression level in lung cancer patients is suppressed as compared to a reference level. The reference level is a level that can be used as a reference for comparison with the level of a complement C3dg molecule in a biological sample derived from a lung cancer patient. When screening for lung cancer or early diagnosis of lung cancer is performed using the method according to the present invention, the level of a complement C3dg molecule in biological samples derived from normal subjects is usually used as the reference level.

In the method according to the present invention, a biological sample to be subjected to analysis is prepared, and then the measured level of a complement C3dg molecule as a lung cancer marker is acquired from the biological sample. An evaluation is made as to whether the measured level is high or low based on the reference level of the lung cancer marker. When the measured level is evaluated as being significantly lower than the reference level, the measured level can be regarded as an indicator indicating that it is highly possible that an individual as a source of the biological sample is affected with lung cancer.

The biological sample to be subjected to analysis may be a biological sample which is derived from an individual who is a subject to be recognized for affecting lung cancer. For example, the biological sample may be a tissue sample or an extract thereof, a body fluid such as blood or serum, or an excretion such as sputum or urine.

The biological sample may be appropriately subjected to pretreatment by those skilled in the art before acquiring the measured level of a complement C3dg molecule. Specific examples of the pretreatment include desalination, cartridge purification, purification using an anti-C3 antibody, and enzymatic digestion (especially, tryptic digestion).

Basically, there is not a large difference in the total amount of complement C3 and its fragments between normal subjects and lung cancer patients. Therefore, in the method according to the present invention, the measured level of a complement C3dg molecule maybe acquired as an absolute value.

However, from the viewpoint of performing more accurate analysis, the measured level of a complement C3dg molecule is preferably acquired as a value relative to the measured level of a complement C3 full-length molecule. This makes it possible to more accurately distinguish a difference between lung cancer patients and normal subjects without being influenced by slight variation in the total amount of complement C3 and its fragments between individuals. In this case, the reference level used for comparison is also a value relative to the reference level of a complement C3 full-length molecule.

In order to acquire the measured level of a complement C3dg molecule, a method may be used in which a complement C3dg molecule whose level should be measured can be distinguished from a C3 full-length molecule, another C3 fragment molecules, or the like.

One example of the method for acquiring the measured level of a complement C3dg molecule includes a method in which a biological sample is subjected to a separation step to separate complement C3dg and the separated complement C3dg molecule is measured.

A method for separating a complement C3dg molecule may be, for example, a method based on a molecular sieving effect. The separation method based on a molecular sieving effect is well-known to those skilled in the art, and is not particularly limited, and examples thereof include electrophoresis, ultrafiltration, chromatography, and the like. Specifically, polyacrylamide gel electrophoresis, centrifugal filtration of low molecular weight proteins, or high-performance liquid chromatography is preferably used.

Examples of a method for measuring the separated complement C3dg molecule include an examination based on biospecific affinity and a quantitative method using mass spectrometry.

The examination based on biospecific affinity is well-known to those skilled in the art and is not particularly limited, but is preferably an immunoassay. Specific examples of the immunoassay include competitive and noncompetitive assay systems such as western blotting, radioimmunoassay, ELISA, sandwich immunoassay, immunoprecipitation method, precipitation reaction, immunodiffusion method, immunoagglutination measurement, complement-binding reaction analysis, immunoradiometric assay, fluoroimmunoassay, and protein A immunoassay. In the immunoassay, the presence of an antibody that binds to a complement C3dg molecule separated from the biological sample is detected. The antibody to be used may be a substance that can form an immunocomplex with complement C3dg, and therefore may be an antibody whose epitope is at least all or part of a complement C3dg sequence. Specifically, the antibody may be an anti-complement C3dg antibody (i.e., an antibody whose epitope is all or only part of a complement C3dg sequence) or an anti-complement C3 polyclonal antibody (i.e., a polyclonal antibody against a complement C3 full-length molecule, which can bind to a complement C3dg molecule).

The mass spectrometry is also a method well-known to those skilled in the art, and is not particularly limited. Examples of a method for introducing a sample into an instrument include connection to a separation device for high-performance liquid chromatography or the like, dropping of a sample to a stainless steel plate, and immersion of a probe in a sample. Examples of a method for ionizing the introduced sample include an electrospray ionization (ESI) method, a matrix assisted laser desorption/ionization (MALDI) method, and the like. Examples of an instrument for measuring the mass of ions include quadrupole-type, ion trap-type, time-of-flight (TOF)-type, Fourier transform ion cyclotron resonance instruments may be used singly or in combination. Those skilled in the art may perform mass spectrometry by appropriately selecting the most suitable combination from these various options.

When the measured level of a complement C3dg molecule is acquired as an amount relative to the measured level of a complement C3 full-length molecule in the above method, a complement C3 full-length molecule separated from the biological sample is also measured in the same manner. An antibody used to measure a complement C3 full-length molecule may be a substance that can form an immunocomplex with a complement C3 full-length molecule, and can be the same as that used to detect a complement C3dg molecule.

Another example of the method for acquiring the measured level of a complement C3dg molecule includes a method using mass spectrometry. This method is preferred in that, unlike the above-described method, the step of separating complement C3dg is not always necessary. The mass spectrometry used in this method is not particularly limited, and may be, for example, one using a mass spectrometer equipped with a MALDI ion source. Specific examples of such a mass spectrometer include a MALDI-TOF (matrix assisted laser desorption/ionization-time of flight)-type mass spectrometer, a MALDI-IT (matrix assisted laser desorption/ionization-ion trap)-type mass spectrometer, a MALDI-IT-TOF (matrix assisted laser desorption/ionization-ion trap-time of flight)-type mass spectrometer, a MALDI-FTICR (matrix assisted laser desorption/ionization-Fourier transform ion cyclotron resonance)-type mass spectrometer, and the like.

In this method, a complement C3 full-length molecule and a complement C3dg molecule can be detected at the same time by subjecting a biological sample to mass spectrometry. Therefore, the measured level of a complement C3dg molecule can be acquired as the amount (mass peak intensity) of the detected complement C3dg molecule relative to the amount (mass peak intensity) of the detected complement C3 full-length molecule. However, in this method, it is not always necessary to detect a complement C3 full-length molecule and a complement C3dg molecule at the same time, and an aspect in which only a complement C3dg molecule is detected is also acceptable.

Alternatively, the measured level of a complement C3dg molecule can be acquired by an internal standard method in which a standard sample having a known concentration is contained in a biological sample to be subjected to mass spectrometry.

### [3. Lung Cancer Detection Kit]

A lung cancer detection kit according to the present invention includes an anti-complement C3dg antibody (i.e., an antibody whose epitope is all or only part of a complement C3dg sequence).

Further, the lung cancer detection kit according to the present invention may further include an item for separating a complement C3dg molecule from a biological sample. Such an item can be appropriately determined by those skilled in the art from the viewpoint of, for example, performing the separation method based on a molecular sieving effect, and examples thereof include a carrier for separation, a buffer, a stain liquid, and the like.

### EXAMPLES

Hereinbelow, the present invention will be specifically described with reference to example, but the present invention is not limited to the following example.

The following test was performed on a total of 30 cases including 10 cases of normal subjects (Normal), 10 cases of early-stage lung cancer patients (Lung cancer stage I-II), and 10 cases of advanced lung cancer patients (Lung cancer stage IIIb-IV).

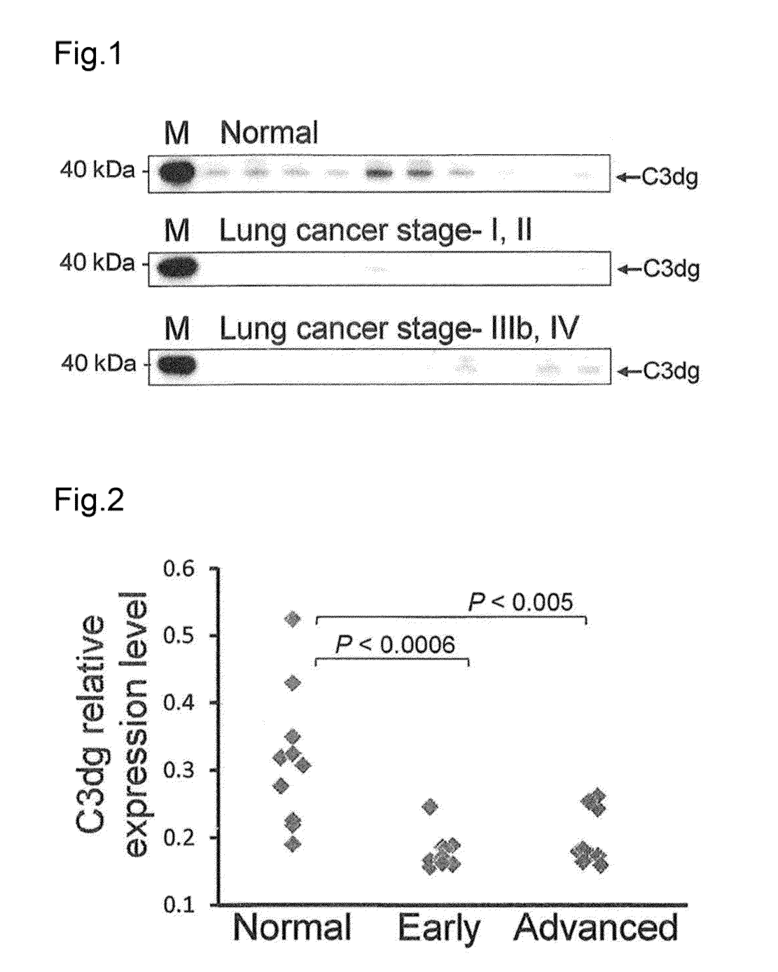

Serum samples of 0.5 μL collected from specimens of the 30 cases were each diluted 100-fold with a sample buffer for polyacrylamide gel electrophoresis (50 mM Tris-HCl pH 6.8, 10% glycerol, 50 mM dithiothreitol, 1.6% SDS, 0.04% Coomassiebrilliant blue G-250), and the diluted serum samples of 20μL were subjected to electrophoresis using NuPAGE Bis-Tris 4-12% polyacrylamide gel. Proteins separated in the gel were directly transferred to a PVDF (PolyVinylidene Difluoride) membrane, and subjected to western blotting using an anti-complement C3 polyclonal antibody (Sigma GW20073F) to visualize protein fragments derived from C3. The results of C3dg fragment detection by western blotting are shown in FIG. 1. In FIG. 1, M represents a molecular weight size marker. FIG. 2 is a graph obtained by plotting C3dg relative expression level detected in FIG. 1. As shown in FIG. 2, the blood concentration of complement C3dg is significantly reduced in the lung cancer cases (especially, in the early-stage lung cancer cases) as compared to the normal subjects (Normal) (advanced lung cancer (Advanced): P<0.005, early-stage lung cancer (Early): P<0.0006).

1. A lung cancer marker comprising a complement C3dg molecule.

2. A method for analyzing a. lung cancer marker comprising the steps of:
measuring a level of a complement C3dg molecule as a lung cancer marker in a biological sample to acquire a measured level of the complement C3dg molecule; and
making an evaluation as to whether the measured level is high or low based on a reference level, of the complement C3dg molecule.

3. The method according to claim 2, wherein the measured level of the complement C3dg molecule is a value relative to a measured level of a complement C3 full-length molecule in the biological sample, and
wherein the reference level of the complement C3dg molecule is a value relative to a reference level of the complement C3 full-length molecule.

4. The method according to claim 2, further comprising the step of subjecting the biological sample to a separation step to obtain the separated complement C3dg,
wherein in the step of acquiring the measured level of the complement C3dg molecule, the complement C3dg molecule is measured using an antibody whose epitope is at least all or part of a complement C3dg sequence.

5. The method according to claim 3, wherein in the step of acquiring the measured level of the complement C3dg molecule, the complement C3 full-length molecule and the complement C3dg molecule are detected at the same time by mass spectrometry to determine an amount of the detected complement C3dg molecule relative to an amount of the detected complement C3 full-length molecule.

6. The method according to claim 2, wherein in the step of acquiring the measured level of the complement C3dg molecule, the complement C3dg molecule is detected by mass spectrometry.

7. (canceled)


SEQUENCE LISTING (1 sequence)

SEQ ID NO: 1 - protein, 349 aa, human
EGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR